SEQUENCE LISTING (4 sequences)

SEQ ID NO: 1 - DNA, 22 bp, Unknown
  Primer
ggtaccatgtcatccaggcctc

SEQ ID NO: 2 - DNA, 30 bp, unknown
  Primer
ggcgatatcatagcctctgtcccaggcaag

SEQ ID NO: 3 - DNA, 29 bp, unknown
  Primer
ggcgatatcggaggctatacagacccaag

SEQ ID NO: 4 - DNA, 20 bp, Unknown
  Primer
ggaggctatacagacccaag

(19) United States
(12) Patent Application Publication
(10) Pub. No.: US 2008/0125364 A1
(43) Pub. Date: May 29, 2008

(54) Methods and Compositions for Treating Epithelial Cancers

(76) Inventors: Asma Nusrat, Atlanta, GA (US); Randall J. Msrny, Menlo Park, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/793,731

(22) PCT Filed: Dec. 28, 2005
(86) PCT No.: PCT/US05/47103

§ 371 Date: Dec. 11, 2007

Related U.S. Application Data
(60) Provisional application No. 60/639,824, filed on Dec. 28, 2004.

(51) Int. Cl. A61K 38/00; C12Q 1/20; C12Q 1/68; A61P 37/00; C12N 5/04; A61K 31/70

(52) U.S. Cl. 514/12; 435/29; 435/6; 435/375; 514/44

(57) ABSTRACT

Methods and compositions for modulating tight junction formation are provided.

### CROSS-REFERENCE TO RELATED APPLICATION

This application is related to and claims the benefit of U.S. Provisional Patent Application No. 60/639,824 filed Dec. 28, 2004, the complete disclosure of which is incorporated herein by reference in its entirety.

### BACKGROUND

1. Technical Field

The present disclosure is generally related to methods and compositions for treating or preventing cancer, in particular to methods and compositions for treating epithelial cancers.

2. Related Art

A crucial function of epithelial cells is in the maintenance of a selective and regulated barrier separating the external from internal environments. The epithelial barrier includes an apical junctional complex (AJC), which is important in determining polarity and barrier properties of epithelial cells, and regulating intercellular adhesion and paracellular permeability in epithelial cells. The AJC is comprised of an occluding junction, or “tight junction” (TJ) and adherens junction (AJ). TJs form a primary barrier to the diffusion of solutes through the paracellular pathway located between adjacent epithelial cells and form a functional fence between the apical and basolateral plasma membrane domains. In addition to their role in barrier function and maintenance of cell polarity, TJs are involved in signal transduction pathways linked to cell growth and proliferation. Because disruption of barrier function, cell polarity, and cell growth and proliferation are common characteristics of epithelial cancers, TJ proteins appear to be attractive candidates for the study of events associated with epithelial oncogenic transformation.

TJ complexes are comprised of transmembrane and cytoplasmic (“scaffolding”) proteins, regulatory enzymes, and transcription factors. Transmembrane protein members include occludin (Furuse et al., 1993), claudins (Furuse et al., 1998a) and junctional adhesion molecules (JAMs) (Martin-Padura et al., 1998). Various cytosolic plaque proteins, such as zonulae occludins (ZO-1, ZO-2 and ZO-3) have been shown to act as scaffolding elements via their ability to bind directly to occludin (Itoh et al., 1997) and claudins (Itoh et al., 1999) as well as to actin (Wittchen et al., 1999). In addition to these scaffold proteins, regulatory enzymes such as atypical protein kinase C (PKC) isotypes, protein kinase A and Rho-like GTPases are found at TJs along with transcription factors such as ZONAB and huASH1 (Matter & Balda, 2003). This multitude of transmembrane proteins, scaffold proteins, regulatory enzymes, and transcription factors together forms a dynamic complex at the TJ that is involved in regulation of barrier function, cell polarity, growth, and proliferation.

Occludin, with a molecular mass of ˜65 kDa, was first identified and characterized as an integral membrane protein localized at TJ strands in chicken (Furuse et al., 1993) and subsequently in various mammalian species (Ando-Akatsuka et al., 1996). Hydropathy plot analysis suggests occludin to be comprised of four transmembrane domains with a long C-terminal cytoplasmic tail and a shorter N-terminal cytoplasmic domain. This proposed topography positions two extracellular loops and one short intracellular turn between the four transmembrane segments. Evidence suggests that occludin is directly involved in TJ barrier function (Balda et al., 1996), in TJ fence function, and in cell adhesion events (Van Itallie & Anderson, 1997). In addition, our previous study suggests that occludin may play a role in rectifying phenotypic changes associated with oncogenic transformation in epithelial cells (Li & Mrsny, 2000).

Various studies have investigated how specific occludin domain deletions or substitutions affect its localization at TJs and its role in barrier function (Balda et al., 1996; Chen et al., 1997; Furuse et al., 1994). Overexpression of mutant forms of occludin, for example, has been shown to affect both barrier and fence function in cultured epithelial cells (Bamforth et al., 1999). Synthetic peptides corresponding to the extracellular loops of occludin were observed to disrupt TJs and inhibit cell adhesion (Lacaz-Vieira et al., 1999; Wong & Gumbiner, 1997). These studies largely agree that occludin plays a central role in TJ barrier and fence functions and that mutation of specific occludin domains affects its localization and function in cells.

Disruption of functional TJ structures is common feature of many human epithelial cancers. Downregulation of specific TJ proteins has been shown to correlate with staging, invasiveness, and metastatic potential in various forms of cancer (Hoover et al., 1998; Tobioka et al., 2004). In endometrial cancers, for example, down-regulation of occludin was shown to correlate with tumor grade, invasiveness, and metastasis, and occludin expression was also observed to decrease in poorly differentiated gastrointestinal adenocarcinomas (Kimura et al., 1997). Besides occludin, other TJ associated proteins such as ZO-1 and claudins have been shown to be important indicators of malignant potential. In some breast cancers, ZO-1 expression was shown to decrease in more malignant forms (Hoover et al., 1998), and expression of claudin-7 was shown to correlate with histological grade (Kominsky et al., 2003). In addition to TJ proteins serving as prognostic indicators, there is mounting evidence to suggest that introduction of TJ proteins into cancer cells can prevent tumor invasion and metastasis. For example, over-expression of claudin-4 has been observed to decrease invasiveness of pancreatic cancer cells in vitro and to decrease metastases in animal models (Michl et al., 2003).

Previous studies (Li & Mrsny, 2000) have demonstrated that overexpression of constitutively active oncogenic Raf1 transformed rat epithelial Pa4 cells into an oncogenic phenotype with downregulation of the TJ protein occludin; whereas, introduction of exogenous occludin into Raf1-transformed cells was observed to rescue the epithelial phenotype and induce reassembly of functional TJs. Further work was needed to investigate and determine the effect of occludin on an oncogenic phenotype in cells.

### SUMMARY

Aspects of the present disclosure generally provide compositions and methods for modulating tight junction formation in cells, particularly transformed cells. Other aspects provide methods for identifying modulators of tight junction formation.

One aspect provides a method for identifying modulators of cellular tight junctions by contacting transformed epithelial or transformed endothelial cells with a test compound, determining formation of functional tight junctions by the transformed cells contacted with the test compound, and selecting the test compound that increases formation of tight junctions compared to a control compound, wherein the test compound increases formation of tight junctions by modulating occludin activity of the transformed cells.

Another aspect provides a method for identifying modulators of epithelial to mesenchymal transformation by contacting occludin or a fragment thereof with a test compound, determining whether the test compound interacts directly or indirectly with occludin or the fragment thereof, and selecting the test compound that interacts with occludin's second loop and reverses epithelial to mesenchymal transformation phenotype changes in transformed cells.

Still another aspect provides a method for modulating Raf1 induced transformation by contacting a cell transformed by Raf1 with a composition that forces expression of occludin in the transformed cell, reversing epithelial to mesenchymal transformation phenotype changes due to Raf1.

Yet another aspect provides a method of treating epithelial or endothelial cell transformation by contacting a transformed epithelial or endothelial cell with a composition comprising an occludin modulator in an amount sufficient to reverse epithelial to mesenchymal transformation phenotype changes.

Other aspects provide compositions for modulating tight junctions. One aspect provides a composition for treating skin cancer comprising a pharmaceutically acceptable carrier or excipient and a vector encoding a recombinant occludin polypeptide including a second loop and carboxy terminus of occludin in an amount sufficient to reverse epithelial to mesenchymal transformation phenotype changes due to Raf1 and.

Another aspect provides a composition for treating epithelial or endothelial cell transformation comprising an occludin modulator in an amount effective to reverse epithelial to mesenchymal transformation phenotype changes due to Raf1.

### BRIEF DESCRIPTION OF THE FIGURES

FIG. 1A is a panel of phase-contrast micrographs of subconfluent cultures of Pa4 and Pa4-Raf 1 and Occ cells.

FIG. 1B is a panel of immunofluorescence micrographs showing labeling of Occludin, claudin-1, ZO-1 and E-cadherin in Pa4, Pa4-Raf1 and Occ cells.

FIG. 1C is a panel of immunoblots illustrating the changes in junctional protein expression in Raf-1 transformed Pa4 cells.

FIG. 1D is a line graph of transepithelial resistance measurements in Pa4, Pa4-Raf1 and Occ cells grown on permeable transwell filters.

FIG. 2A is a diagram of full-length occludin showing regions corresponding to constructs encoding specific occludin domain deletions that were expressed in Raf1-transformed Pa4 cells. All constructs contain a C-terminal myc-tag. Occ is full-length human occludin; Occ ΔAN lacks the short N-terminal tail and both extracellular loops; Occ ΔC lacks C-terminal cytoplasmic domain; OccΔL1 lacks the first extracellular loop; OccΔL2 lacks the second extracellular loop.

FIG. 2B is a panel of micrographs of cells expressing various Occ mutations. The top panel is a series of phase-contrast images showing that cells expressing OccΔN, OccΔC, or OccΔL2 exhibit a fibroblast-like morphology, while cells expressing full-length occludin or OccΔL1 acquire the typical cobblestone-like appearance of the parental Pa4 epithelial cells. The bottom panel is a series of immunofluorescence micrographs in Pa4-Raf 1 cells with antibodies against ZO-1 and myc-tagged occludin constructs. Note that the Pa4-Raf1 cells expressing OccΔN, OccΔC, OccΔL2 exhibit diffuse, disorganized ZO-1 and myc staining, while cells expressing full-length occludin or OccΔL1 demonstrate typical staining of ZO-1 and myc-tagged occludin in the lateral membranes of intercellular junctions. Scale bar 10.

FIG. 3A is a panel of immunofluorescence confocal images illustrating occludin, claudin-1, ZO-1 and E-cadherin staining at intercellular junctions in cells expressing the occludin mutant lacking the first loop (OccΔL1). In contrast, occludin, claudin-1, ZO-1 and E-cadherin demonstrate a diffuse, disorganized and intracellular staining pattern in cells expressing the occludin mutant lacking the second loop (OccΔL2).

FIG. 3B is a panel of confocal images in the x-z plane illustrating localization of occludin in Pa4-Raf1 cells. In cells expressing full-length occludin (Occ), occludin is concentrated at the tight junction with minimal lateral membrane staining. In cells expressing occludin lacking the first loop (OccΔL1), occludin also localizes at tight junctions, but slightly more lateral membrane staining is observed. Lastly, cells expressing occludin lacking the second loop (OccΔL2) fail to form a polarized monolayer, with no discernable apical junctional complexes.

FIG. 4 is an immunoblot showing Triton X-100-soluble (S) and insoluble (I) protein fractions prepared from cells expressing occludin mutants lacking the first extracellular loop (OccΔL1) and second extracellular loop (OccΔL2) and compared with extracts from Pa4 cells and Raf1-transformed Pa4 cells (Pa4-Raf1). Protein fractions were immunoblotted for the junctional proteins occludin, claudin-1, E-cadherin, or ZO-1.

FIG. 5A is a line graph showing transepithelial resistance to passive ion flux in Pa4-Raf1 cells expressing full-length occludin or occludin mutants lacking either the first extracellular loop or the second extracellular loop.

FIG. 5B is a bar graph showing paracellular flux of FD-3 (fluorescent dextran; 3000 Da).

FIG. 6A is a panel of photographs of Pa4-Raf1 cells expressing occludin constructs with specific domain deletions plated into 35-mm dishes in soft agar. Size bar 2 mm.

FIG. 6B is a bar graph showing the average number of colonies for each cell line.

FIG. 7A is a panel of representative photographs of nude mice injected with cells expressing wild type, occludin (Occ) or mutants lacking the second loop (OccΔL2).

FIG. 7B is a table showing the average tumor weights from mice injected with each cell line.

FIGS. 8A and B are plots showing a comparison of mRNA abundance levels between Pa4-Raf versus Pa4 and Pa4-Raf versus Pa4-Raf1-Occ.

FIG. 9 is a plot showing a comparison of mRNA abundance level in Pa4-Raf1 to those of Pa4-Raf1-Occ cells.

FIG. 10 is a plot showing a comparison of mRNA abundance level in Pa4-Claudin versus Pa4-Occ cells.

### DETAILED DESCRIPTION

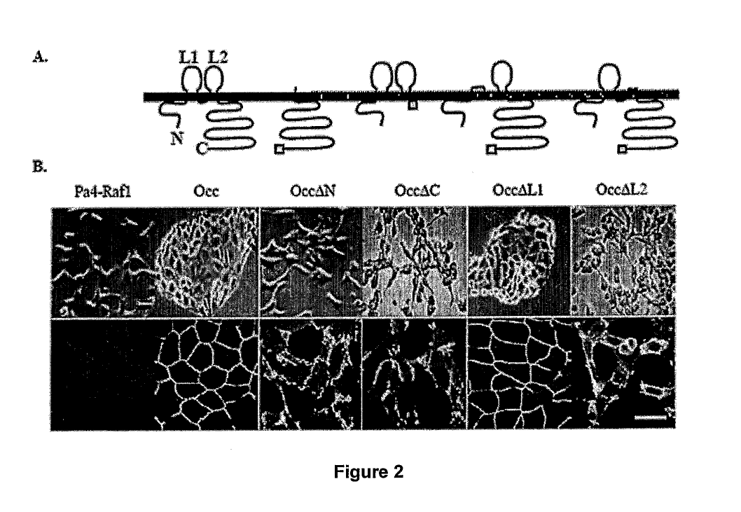

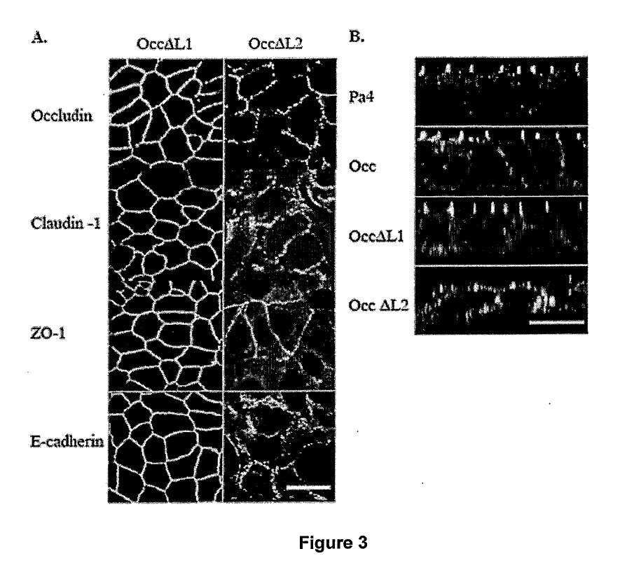

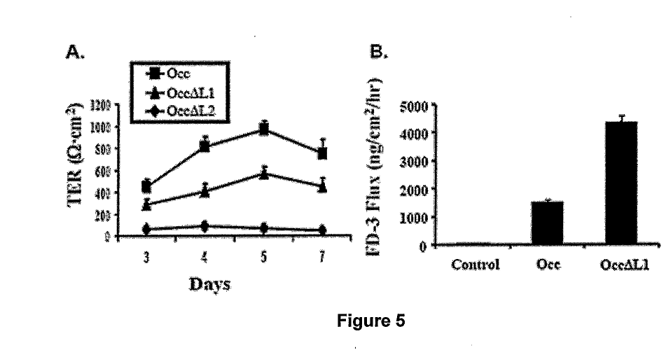

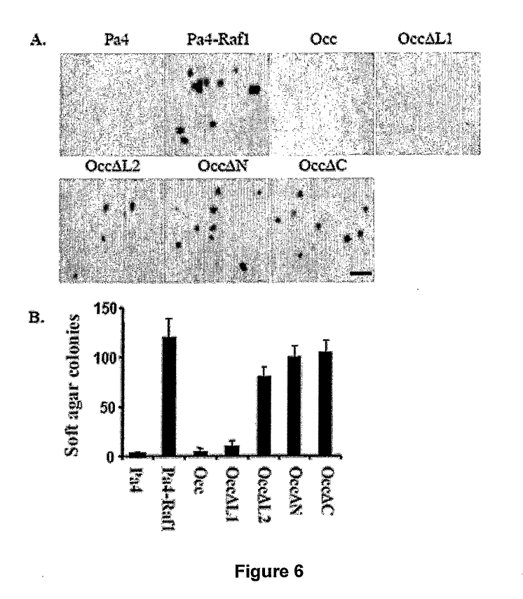

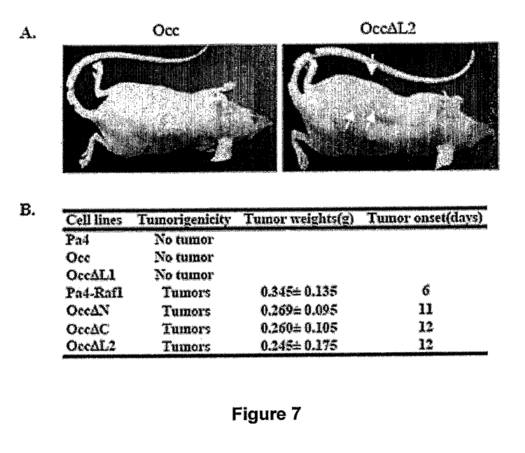

In epithelial cells, TJs are important for regulating paracellular solute flux and cell polarity. Disruption of TJ proteins has been reported in epithelial cancers, but heretofore, the role of TJ proteins in regulation of oncogenic transformation remained largely unexplored. Previous studies have identified an ability of the TJ protein occludin to correct the phenotype of an epithelial cell line (Pa4) transformed by active Raf1 (Li & Mrsny, 2000). Employing a rescue-of-function approach, the instant disclosure demonstrates results of an investigation as to how specific domains of occludin affect its ability to correct Raf1-mediated cell transformation using this same Pa4 cell system. One of the most remarkable findings from the present studies showed that the proposed second extracellular loop of occludin is important in the rescue of epithelial morphology (FIG. 2), assembly of junctional complexes (FIGS. 3 & 4), and TJ barrier function (FIG. 5). In addition, the second loop of occludin is important for reversing Raf1-driven anchorage dependent growth of soft agar in vitro (FIG. 6) and tumor growth in vivo (FIG. 7).

Accumulating evidence suggests that occludin plays an important role in the structure and function in TJs. Overexpression of mutant forms of occludin has been shown to affect both barrier and fence function in cultured epithelial cells (Balda et al., 1996; Bamforth et al., 1999), and synthetic peptides corresponding to the extracellular loops of occludin were observed to disrupt TJs and inhibit cell adhesion (Lacaz-Vieira et al., 1999; Wong & Gumbiner, 1997). In endothelial cells, occludin expression appears to correlate with TJ function in that endothelial cells of the blood-brain barrier express significantly higher levels of occludin than systemic endothelial cells, which have leakier tight junctions (Hirase et al., 1997). Finally, occludin knock-out mice show a complex phenotype that includes retarded growth and a variety of tissue-specific abnormalities, suggesting that the function of occludin is important (Saitou et al., 2000). Together these studies provide strong evidence that occludin is key a component of the TJ, critical for epithelial and endothelial cell function.

Various studies have investigated the effect of specific mutations on occludin localization and TJ function. Although many of these studies agree that the occludin protein is a critical component of the TJ, some inconsistencies have arisen regarding whether specific occludin domains are required for its targeting and formation of intact, functional TJs. For instance, occludin constructs lacking the entire C-terminal tail were observed to localize at the TJs (Balda et al., 1996; Chen et al., 1997), and the C-terminal tail alone does not appear to be sufficient for localization (Matter & Balda, 1998). These results, however, are at variance with another study that reported that a C-terminally truncated occludin construct failed to localize at TJs (Furuse et al., 1994). In addition, occludin constructs lacking the first extracellular loop were observed to localize successfully at TJs but constructs lacking the second loop or both extracellular domains did not (Bamforth et al., 1999; Medina et al., 2000). Thus it appears that the extracellular domains of occludin, particularly the second loop, is important for its localization in TJs, yet the requirement for the C-terminal tail remains unclear. Subtle differences in the occludin constructs employed and the origin of the cell lines used in these studies may account for some of these apparent inconsistencies. In particular, the presence of endogenous occludin and its binding partners may influence the localization of transfected occludin constructs and their affect on TJ function. A unique aspect of our current study is the use of the Raf1-transformed Pa4 cells. Although Pa4-Raf 1 cells do not express occludin or form junctions, they are derived from well-differentiated Pa4 epithelial cells capable of forming functionally and morphologically intact TJs. This model proved useful for investigating how structural modifications to the occludin protein affected its function in epithelial cells.

Previous studies have shown that administration of a peptide corresponding to the second loop of occludin disrupted transepithelial electrical resistance and resulted in the disappearance of occludin from cell junctions (Wong & Gumbiner, 1997). Thus, the second extracellular loop of occludin appears to be important for concentrating and/or stabilizing the occludin protein within TJ complexes. Other studies have shown that the extracellular domains of occludin are directly involved in cell-cell adhesion (Van Itallie & Anderson, 1997), and homophilic occludin interactions between cells is likely dependent on its extracellular domains (Furuse et al., 1998b). It has been reported (Medina et al., 2000) that occludin lacking the first extracellular loop co-localized with ZO-1 at the TJ, while occludin lacking the second loop was absent from tight junctions. These data support the idea that interactions involving the extracellular domains may be critical for the occludin function in TJs. Consistent with these reports, we observed that expression of occludin protein lacking the second extracellular loop in Pa4-Raf1 cells did not rescue epithelial morphology or restore TJ structure or function. It is interesting, however, that the occludin mutant lacking the first loop localized at TJs (FIG. 2) and promoted assembly of TJ complexes (FIGS. 3 & 4), and yet only partially rescued barrier function (FIG. 5). These results suggest that the first loop may not be required for assembly of occludin into the TJ complex, but the first loop may play a role in establishing and/or maintaining paracellular permeability properties of the TJ.

Claudins, which compose a multigene family, have been shown to be directly involved in barrier function of TJs (Morita et al., 1999; Tsukita & Furuse, 1999). Together, claudins and occludin are thought to constitute the backbone of TJ strands. Recent studies suggest that direct interactions between claudins and occludin contribute to their function. For example, occludin was demonstrated to cooperate with claudin-4 to mediated selective paracellular permeability (Balda et al., 2000). An interaction between occludin and claudins is also suggested by the recruitment of occludin into claudin-induced intramembrane strands in L-cells (Furuse et al., 1998b). In a previous study, it was shown that the extent of full-length occludin expression positively correlated with total claudin-1 protein levels, strongly suggesting an interaction between occludin and claudins. The present study demonstrates that upregulation of claudin-1 by occludin utilizes requires its second extracellular loop, supporting the concept that the extracellular domains of these proteins may interact directly. In interpreting these results, however, it is important to consider that up-regulation and stabilization of claudin-1 at TJs may in fact be mediated indirectly by other TJ-associated proteins such as ZO-1, whose localization at TJs is also affected by occludin expression.

The molecular mechanism by which occludin inhibits Raf1-induced transformation is not well understood. Disclosed hereinbelow are microarray experiments in which overexpression of wild type occludin in Pa4-Raf1 cells resulted in dramatic changes in gene expression. This finding suggests that occludin-mediated rescue of the epithelial phenotype in Raf1-transformed cells may involve regulation at the transcriptional level. Although little is known about the relationship between TJ proteins and transcription factors, growing evidence suggests that TJ proteins play a role in regulating gene expression, perhaps through TJ-associated adaptor proteins such as ZO-1. For example, regulation of ErbB2 by ZO-1 is thought to involve sequestration of the transcriptional factor ZONAB (Balda & Matter, 2000), and interaction of ZO-1 with ZONAB appears to play a role in CDK4-mediated regulation of cell growth and proliferation (Balda et al., 2003). Recently, interactions between ZO-2 and the transcription factors Fos, Jun and C/EBP have been demonstrated to regulate gene expression via AP-1 (Betanzos et al., 2004). Another transcription factor associated with TJs is ASH1 (Nakamura et al., 2000), but less is known about how TJ proteins affect ASH-regulated gene expression. Given the dramatic effect of occludin in reversing the oncogenic phenotype in PA4-Raf1 cells, it is likely that transcriptional regulation plays a role in this process. Future studies may consider how occludin expression and localization at the TJ might directly affect specific transcriptional factors that localize in TJs such as ASH and others known to interact with ZO-1 and ZO-2.

Evidence is now accumulating that expression of the TJ protein occludin can suppress Raf1-transformation of epithelial cells in vitro and in vivo. The present disclosure now presents direct evidence to show that the second loop of occludin is important for reversing the epithelial to mesenchymal transformation (EMT) phenotype changes associated with activation of Raf1. EMT reversal by occludin is also associated with formation of functional TJs and re-acquisition of a polarized phenotype characteristic of functional epithelia. Importantly, these results demonstrate that TJs play a pivotal role in regulation of epithelial carcinomas and suggest that the TJ protein occludin participates in a functional dynamic with the Raf1 oncogene to control events associated with EMT. Disruption of TJs is considered an important indicator of invasion and metastasis in human epithelial cancers, and TJ proteins such as occludin may prove to be valuable molecular targets for diagnosis and treatment such cancers.

### Screening

The disclosure also provides methods for identifying modulators of occludin activity, modulators of cell transformation, and modulators of tight junction formation. As used herein the term “test compound” or “modulator” refers to any molecule that may potentially inhibit or enhance the formation of tight junctions, in particular occludin mediated tight junction formation. Representative modulators mimic or increase activity or expression of occludin or a fragment thereof, such as the second loop of occludin optionally including the carboxy terminus of occludin. The test compound or modulator can be a protein or fragment thereof, a small molecule, or even a nucleic acid molecule. Some test compounds and modulators can be compounds that are structurally related to occludin polypeptides. The disclosure contemplates using lead compounds to help develop improved compounds and includes not only comparisons with known inhibitors and activators, but predictions relating to the structure of target molecules such as occludin and in particular the second loop of occludin.

One embodiment provides a method for identifying modulators of tight junction formation including assaying the formation of tight junctions by an occludin polypeptide, a homolog, or fragment thereof in the presence of a test compound, and selecting the test compound that promotes or interferes with occludin-mediated tight junction formation as compared to a control compound.

In another embodiment, small molecule libraries that are believed to meet the basic criteria for useful drugs can be screened to identify useful compounds. Screening of such libraries, including combinatorially generated libraries (e.g., expression libraries), is a rapid and efficient way to screen large number of related (and unrelated) compounds for activity. Combinatorial approaches also lend themselves to rapid evolution of potential drugs by the creation of second, third, and fourth generation compounds modeled of active, but otherwise undesirable compounds.

Test compounds may include fragments or parts of naturally occurring compounds, or may be found as active combinations of known compounds, which are otherwise inactive. Compounds isolated from natural sources, such as animals, bacteria, fungi, plant sources, (including leaves and bark), and marine samples can be assayed as candidates for the presence of potentially useful pharmaceutical agents. It will be understood that the pharmaceutical agents to be screened could also be derived or synthesized from chemical compositions or man-made compounds. Thus, it is understood that the test compound identified by embodiments of the present disclosure may be a peptide, polypeptide, polynucleotide, small molecule inhibitor, small molecule inducer, organic or inorganic, or any other compound that may be designed based on known inhibitors or stimulators.

Other suitable modulators include antisense molecules, catalytic nucleic acids such as ribozymes and antibodies (including single chain antibodies), each of which would be specific for occludin, in particular specific for the second loop of occludin. For example, an antisense molecule that binds to a translational or transcriptional start site, or splice junctions, is within the scope of a test compound.

In addition to the modulating compounds initially identified, other sterically similar compounds may be formulated to mimic the key portions of the structure of the modulators, for example the second loop of occludin. Such compounds, which may include peptidomimetics of peptide modulators, may be used in the same manner as the initial modulators.

An inhibitor or activator according to the present disclosure may be one which exerts its inhibitory or activating effect upstream, downstream, directly, or indirectly on occludin mediated tight junction formation. In one embodiment, the inhibition or activation by an identified modulator results in the modulation of occludin biological activity or expression as compared to that observed in the absence of the added test compound.

### Screening for Modulators of Tight Junction Formation

Embodiments of the present disclosure include methods for identifying modulators of the function, expression, or bioavailability of occludin, in particular the function of occludin in the formation of tight junctions. The modulator may modulate occludin-mediated tight junction formation directly or indirectly. Direct modulation refers to a physical interaction between the modulator and occludin, an occludin receptor, or an occludin binding site, for example binding of the modulator to a region of occludin such as the second loop of occludin. Indirect modulation of occludin-mediated tight junction formation can be accomplished when the modulator physically associates with a cofactor, second protein, or second biological molecule that interacts with occludin either directly or indirectly. Additionally, indirect modulation would include modulators that affect the expression or the translation of RNA encoding occludin.

In some embodiments, the assays can include random screening of large libraries of test compounds. Alternatively, the assays may be used to focus on particular classes of compounds suspected of modulating the function or expression of occludin in epithelial or endothelial cells, tissues, organs, or systems.

Assays can include determinations of occludin expression, protein expression, protein activity, or binding activity. Other assays can include determinations of nucleic acid transcription or translation, for example mRNA levels, mRNA stability, mRNA degradation, transcription rates, and translation rates, particularly of polypeptides involved in tight junction formation.

In one embodiment, the identification of a tight junction modulator is based on the function of occludin in the presence and absence of a test compound. The test compound or modulator can be any substance that alters or is believed to alter the function of occludin, in particular the function of occludin in the formation of tight junctions. Typically, a modulator will be selected that reduces, eliminates, or mitigates occludin mediated tight junction formation. However, for treatment purposes, modulators can be selected that increase or reduce tight junction formation.

One exemplary method includes contacting occludin with at least a first test compound, and assaying for an interaction between occludin and the first test compound. The assaying can include determining occludin-mediated tight junction formation.

Specific assay endpoints or interactions that may be measured in the disclosed embodiments include, but are not limited to, assaying for tight junction formation, ion permeability, transepithelial electrical resistance, occludin down or up regulation or turnover. These assay endpoints may be assayed using standard methods such as FACS, FACE, ELISA, Northern blotting and/or Western blotting. Moreover, the assays can be conducted in cell free systems, in isolated cells, in genetically engineered cells, in immortalized cells, or in organisms including transgenic animals.

Other screening methods include using labeled occludin to identify a test compound. Occludin can be labeled using standard labeling procedures that are well known and used in the art. Such labels include, but are not limited to, radioactive, fluorescent, biological and enzymatic tags.

Another embodiment provides a method for identifying a modulator of occludin expression by determining the effect a test compound has on the expression of occludin in skin, epithelial, or endothelial cells. For example, skin cells expressing occludin can be contacted with a test compound. Occludin expression can be determined by detecting occludin protein expression or occludin mRNA transcription or translation. Suitable cells for this assay include, but are not limited to, immortalized cell lines, primary cell culture, or cells engineered to express occludin, for example PA4 cells. Compounds that modulate the expression of occludin, in particular those that increase the expression or bioavailability of occludin, can be selected.

### In Vitro Assays

Another embodiment provides for in vitro assays for the identification of occludin modulators. Such assays generally use isolated molecules and can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule, for example a nucleic acid encoding occludin, in a specific fashion is strong evidence of a related biological effect. Such a molecule can bind to a occludin nucleic acid and modulate expression of occludin, for example upregulate expression of occludin. The binding of a molecule to a target may, in and of itself, be inhibitory due to steric, allosteric or charge-charge interactions, or it may downregulate or inactivate occludin. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### Cell Assays

Other embodiments include methods of screening compounds for their ability to modulate occludin in cells. Various cell lines can be utilized for such screening assays, including cells specifically engineered for this purpose. Suitable cells include, but are not limited to, PA4 cells, endothelial cells, or epithelial cells. Cells can also be engineered to express occludin or a modulator of occludin or a combination of both occludin and a modulator of occludin. Furthermore, those of skill in the art will appreciate that stable or transient transfections, which are well known and used in the art, may be used in the disclosed embodiments.

For example, introducing an expression vector into a cell can generate a transgenic cell comprising the expression vector. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome-mediated transfection, DEAE dextran mediated transfection, electroporation, ballistic bombardment, and the like. Alternatively, cells may be simply transfected with the disclosed expression vector using conventional technology described in the references and examples provided herein. The host cell can be a prokaryotic or eukaryotic cell, or any transformable organism that is capable of replicating a vector and/or expressing a heterologous gene encoded by the vector. Numerous cell lines and cultures are available for use as a host cell, and they can be obtained through the American Type Culture Collection (ATCC), which is an organization that serves as an archive for living cultures and genetic materials, which organization's website can be found on worldwide web at the follow address: atcc.org.

A host cell can be selected depending on the nature of the transfection vector and the purpose of the transfection. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE, La Jolla, Calif.). Alternatively, bacterial cells such as E. coli LE392 could be used as host cells for phage viruses. Eukaryotic cells that can be used as host cells include, but are not limited to, yeast, insects and mammals. Examples of mammalian eukaryotic host cells for replication and/or expression of a vector include, but are not limited to, HeLa, NIH3T3, Jurkat, 293, Cos, CHO, Saos, and PC12. Examples of yeast strains include, but are not limited to, YPH499, YPH500 and YPH501. Many host cells from various cell types and organisms are available and would be known to one of skill in the art. Similarly, a viral vector may be used in conjunction with either an eukaryotic or prokaryotic host cell, particularly one that is permissive for replication or expression of the vector.

Depending on the assay, culture may be required. The cell is examined using any of a number of different physiologic assays. Alternatively, molecular analysis may be performed, for example, by looking at protein expression, mRNA expression (including differential display of whole cell or polyA RNA), and others.

### In vivo Assays

In vivo assays involve the use of various animal models, including non-human transgenic animals that have been engineered to have specific defects, or carry markers that can be used to measure the ability of a test compound to reach and affect different cells within the organism. Due to their size, ease of handling, and information on their physiology and genetic make-up, mice are a preferred embodiment, especially for transgenic animals. However, other animals are suitable as well, including but not limited to rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more test compounds are administered to an animal, and the ability of the test compound(s) to alter one or more characteristics, as compared to a similar animal not treated with the test compound(s), identifies a modulator. The characteristics may be any of those discussed above with regard to the function of a particular compound (e.g., enzyme, receptor, hormone) or cell (e.g., growth or regeneration), or instead a broader indication nerve cell regeneration, axonal growth or regeneration, or the like.

Other embodiments provide methods of screening for a test compound that modulates the function of occludin. In these embodiments, a representative method generally includes the steps of administering a test compound to the animal and determining the ability of the test compound to reduce one or more characteristics of epithelial to mesenchymal transformation.

Treatment of these animals with test compounds will involve the administration of the compound, in an appropriate form, to the animal. Administration will be by any route that could be utilized for clinical or non-clinical purposes, including, but not limited to, oral, nasal, buccal, or even topical. Alternatively, administration may be by intratracheal instillation, bronchial instillation, and intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### Pharmaceutical Compositions

Pharmaceutical compositions and dosage forms of the disclosure include a pharmaceutically acceptable salt of disclosed compositions or compounds or a pharmaceutically acceptable polymorph, solvate, hydrate, dehydrate, co-crystal, anhydrous, or amorphous form thereof. Specific salts of disclosed compounds include, but are not limited to, sodium, lithium, potassium salts, and hydrates thereof.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. A parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that include primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure include a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, more preferably in an amount of from 50 mg to 500 mg, even more preferably in an amount of from about 30 mg to about 100 mg.

Additionally, the compounds and/or compositions can be delivered using lipid- or polymer-based nanoparticles. For example, the nanoparticles can be designed to improve the pharmacological and therapeutic properties of drugs administered parenterally (Allen, T. M., Cullis, P. R. Drug delivery systems: entering the mainstream. Science. 303(5665):1818-22 (2004)).

Topical dosage forms of the disclosure include, but are not limited to, creams, lotions, ointments, gels, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms including a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art. See, e.g., Remington's Pharmaceutical Sciences, 18th Ed., Mack Publishing, Easton, Pa. (1990).

Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of a tight junction or occludin modulator of the disclosure. For example, penetration enhancers can be used to assist in delivering the active ingredients to or across the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of a tight junction modulator can be used to further adjust the properties of the resulting composition.

### DEFINITIONS

The term “organism” or “host” refers to any living entity comprised of at least one cell. A living organism can be as simple as, for example, a single eukaryotic cell or as complex as a mammal, including a human being.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or a pharmaceutically acceptable salt thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985). Design of Prodrugs, Elsevier; Wang et al. (1999). Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990). Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985). Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr Drug Metab., 1(1):31-48; D. M. Lambert (2000). Rationale and applications of lipids as prodrug carriers, Eur. J. Pharm. Sci., 11 Suppl 2:S15-27; Wang, W. et al. (1999). Prodrug approaches to the improved delivery of peptide drugs. Curr. Pharm. Des., 5(4):265-87.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids. Anti-sense is a polynucleotide that interferes with the function of DNA and/or RNA. Natural nucleic acids have a phosphate backbone, artificial nucleic acids may contain other types of backbones, but contain the same bases.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring, such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: lie, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in Lesk, A. M., Ed. (1988) Computational Molecular Biology, Oxford University Press, New York; Smith, D. W., Ed. (1993) Biocomputing: Infomatics and Genome Projects. Academic Press, New York; Griffin, A. M., and Griffin, H. G., Eds. (1994) Computer Analysis of Sequence Data: Part I, Humana Press, New Jersey; von Heinje, G. (1987) Sequence Analysis in Molecular Biology, Academic Press; Gribskov, M. and Devereux, J., Eds. (1991) Sequence Analysis Primer. M Stockton Press, New York; Carillo, H. and Lipman, D. (1988) SIAM J Applied Math., 48, 1073.

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, ((1970) J. Mol. Biol., 48, 443-453) algorithm (e.g., NBLAST, and XBLAST).

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “treating” includes alleviating the symptoms associated with a specific disorder or condition and/or preventing or eliminating said symptoms.

As used herein, the terms “modulate(s),” “modulating,” “modify,” and/or “modulator” generally refers to the act of directly or indirectly promoting/activating or interfering with/inhibiting a specific function or behavior. For instance, a modulator of cellular tight junctions might activate the barrier function of cells, regulate cell polarity, or cell growth and proliferation, or a modulator of cellular tight junctions might inhibit activate the barrier function of cells, regulate cell polarity, or cell growth and proliferation. In some instances, a modulator may increase and/or decrease a certain activity or function relative to its natural state or relative to the average level of activity that would generally be expected or relative to a current level of activity.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “vector” is used in reference to a vehicle used to introduce a nucleic acid sequence into a cell. A viral vector is virus that has been modified to allow recombinant DNA sequences to be introduced into host cells or cell organelles.

The term “heterologous” means derived from a separate genetic source, a separate organism, or a separate species. Thus, a heterologous antigen is an antigen from a first genetic source expressed by a second genetic source. The second genetic source is typically a vector.

The term “recombinant” generally refers to a non-naturally occurring nucleic acid, nucleic acid construct, or polypeptide. Such non-naturally occurring nucleic acids include combinations of DNA molecules of different origin that are joined using molecular biology technologies, or natural nucleic acids that have been modified, for example that have deletions, substitutions, inversions, insertions, etc. Recombinant also refers to the polypeptide encoded by the recombinant nucleic acid. Non-naturally occurring nucleic acids or polypeptides include nucleic acids and polypeptides modified by man.

### Materials and Methods

### Occludin Deletion Constructs

Occludin deletion constructs were generated by PCR-based mutagenesis using cDNA coding for human occludin as a template. PCR amplifications were performed using the proofreading pfu DNA polymerase (Stratagene) and products were subcloned into the pCR-Blunt II-TOPO vector (Invitrogen). Insert genotypes were confirmed by sequence analysis, and were then cloned into the pcDNA4-myc mammalian expression vector (Invitrogen). A 10-amino acid myc tag was engineered at the C-terminus of each occludin deletion construct to facilitate immunodetection. A truncated form of the first extracellular loop, OccΔL1, was constructed by digesting and ligating two PCR products. The first PCR product was amplified using two primers 5′-GGTACCATGTCATCCAGGCCTC-3′ (SEQ ID NO:1) and 5′-GGCGATATCATAGCCTCTGTCCCAGGCAAG-3′ (SEQ ID NO:2). The resulting PCR product encoded the N-terminal end of occludin, including the first transmembrane domain and the first four amino acids of the first extracellular loop, followed by an engineered EcoRV site. A second PCR product was amplified using two primers 5′-GGCGATATCGGAGGCTATACAGACCCAAG-3′ (SEQ ID NO:3) and 5′-GGAGGCTATACAGACCCAAG-3′ (SEQ ID NO:4) to generate a PCR product containing the C-terminal end of occludin starting with an engineered EcoRV site followed by the second transmembrane domain and the rest of the occludin protein. The two products were cloned into pcDNA4 vector (Invitrogen) and the two inserts were combined using the engineered EcoRV site. The first extracellular loop of human occludin is predicted to include 46 residues encompassing residues D⁹⁰ and R¹³⁵. The truncated first extracellular loop, OccΔL1, contained 13 amino acids D⁹⁰RGYDIGGYTDPR¹³⁵ resulting from deletion of residues 94 to 128 and insertion of residues DI (from the EcoRV site) that are not found in the authentic occludin sequence. The construct containing a truncated form of the second extracellular loop, OccΔL2, was made using the same approach. The second loop of human occludin is predicted to include 48 residues from G¹⁹⁶ to E²⁴³. The truncated second extracellular loop, OccΔL2, contained the amino acids G¹⁹⁶VNPKLVDPQE²⁴³, resulting from deletion of residues 200 to 238 and insertion of residues KL not found in occludin. The construct, OccΔN, includes the N-terminal domain and extracellular loops (amino acids 1-266) and lacks the C-terminal domain. The construct, OccΔC, contains only the C-terminal domain (amino acids 243-522). All these occludin mutants were constructed using same approaches as mentioned above.

### Cell Culture and Generation of Stable Cell Lines

Pa4 cells, a highly differentiated epithelial cell line derived from normal rat parotid acinar epithelium, were cultured in Dulbecco's modified Eagle's/F12 (1:1) medium supplemented with 2.5% fetal bovine serum, insulin (5 μg/ml), transferrin (5 μg/ml), epidermal growth factor (25 ng/ml), hydrocortisone (1.1 μM), and glutamate (5 mM) and were maintained in a humidified atmosphere containing 5% CO₂ at 35° C. Pa4-Raf1 and Occ cells were established as described previously (Li & Mrsny, 2000) and maintained in Dulbecco's modified Eagle's/F12 (1:1, phenol red-free) medium supplemented with 2.5% charcoal-stripped fetal bovine serum plus the above-mentioned factors. G418 (500 μg/ml) was used for selection of Pa4-Raf1 cells while G418 (500 μg/ml) and hygromycin (100 μg/ml) were used for double selection of the Occ cell line. Charcoal-stripped serum was used in maintaining Pa4-Raf1 and Occ stable cell lines to minimize the estrogen level in the culture medium. Pa4-Raf1 cells were transfected with various occludin deletion constructs using a Lipofectamine Plus reagent (Life Technologies). Stable cell lines were selected in G418 (500 μg/ml) and Zeocin (200 μg/ml). Resistant colonies were isolated and maintained in Dulbecco's modified Eagle's/F12 (1:1, phenol red-free) medium supplemented with 2.5% charcoal-stripped fetal bovine serum, G418 (500 μg/ml), Zeocin (200 μg/ml), plus the above-mentioned factors.

Pa4-Raf1-Occ refers to Pa4-Raf1 cells transfected with the full length human occludin gene. These cells have epithelial characteristics (i.e. polarize, develop mature tight junctions and adherens junctions).

### Immunofluorescence Microscopy

Cells grown on glass coverslips or on polycarbonate filters (NK, Costar, Cambridge, Mass., US) with a pore size of 0.4 μm were rinsed with HBSS containing calcium and magnesium (HBSS⁺). Cells were fixed and permeabilized in absolute methanol for 20 min at −20° C., followed by blocking in HBSS⁺ containing 5% normal goat serum (blocking buffer) for 1 h at room temperature and incubation for 60 min with primary antibodies to human occludin, claudin-1, and ZO-1, (Zymed Laboratories, San Francisco, Calif.), E-cadherin (BD Biosciences) in blocking buffer. Cells were then washed, incubated for 60 min with Alexa dye-conjugated secondary antibodies (Molecular Probes, Eugene, Oreg.), followed by rinsing and mounting on slides with PROLONG ANTIFADE medium (Molecular Probes). For double labeling of junctional proteins, cells were fixed and permeabilized in absolute methanol and blocked in blocking buffer. Cells were sequentially stained with two primary antibodies. Donkey anti-goat and anti-rabbit antibodies conjugated to fluorescent red or green Alexa dyes (Molecular Probes) were used for detection of the respective primary antibodies. Stained cells were scanned using a Zeiss LSM510 laser scanning confocal microscope (Zeiss Microimaging Inc., Thornwood, N.Y.) coupled to a Zeiss 100M axiovert and 40× or 63× Pan-Apochromat oil lenses. Fluorescent dyes were imaged sequentially in frame-interlace mode to eliminate crosstalk between channels. Images shown are representative of at least three experiments, with multiple images taken per slide.

### Western Blots and Detergent Solubility Assays

To make Triton X-100-soluble and -insoluble pools, cells were washed twice with PBS and lysed in gold lysis buffer containing 20 mM Tris-HCl, pH 8.0, 137 mM NaCl, 5 mM EDTA, 10% (vol/vol) glycerol, 1% (vol/vol) Triton X-100, 1 mM PMSF, 1 mM aprotinin, 1 mM leupeptin, 1 μM pepstatin A, 1 mM sodium orthovanadate, 1 mM EGTA, 10 mM NaF, 1 mM tetrasodium pyrophosphate, and 100 μM β-glycerophosphate at 4° C. Lysates were incubated at 4° C. for 10 min, and then centrifuged at 15,000×g for 30 min at 4° C. This supernatant was considered the Triton X-100-soluble pool. The pellet was solubilized in 1% SDS and referred to as the Triton X-100-insoluble pool. Protein concentrations were determined by Pierce BCA assay. Equal amounts of protein of cellular lysates (50 μg) were subjected to SDS-PAGE. After electrophoresis, proteins were electroblotted onto PVDF membranes (Bio-Rad Laboratories). Membranes were blocked with 5% milk solution for 1 h before incubation with primary antibodies. HRP-conjugated secondary antibodies and the enhanced chemiluminescence detection system (NEN™ Life Science Products, Inc.) were used to detect bound antibodies.

### Measurement of TER and Paracellular Permeability

For transepithelial electrical resistance (TER) measurements, cells were plated on polycarbonate filters with a pore size of 0.4 μm. A Millicell-ERS volt-ohm meter (Millipore) was used to determine the TER value. All TER values were normalized for the area of the filter and were obtained by subtracting the contribution of the filter and bathing solution. Paracellular permeability to fluoresceinated dextran (FD-3; MW 3000) was assessed according to previously published methods (Sanders et al., 1995). Briefly, cells were grown on polycarbonate filters with a pore size of 0.4 μm. TER was measured and then cells were washed in Hanks balanced salt solution/10 mM HEPES (HBSS⁺) and equilibrated at 37° C. for 10 min on an orbital shaker. Cells were loaded apically with 1 mg/ml FD-3 (Molecular Probes) at time=0. Basolateral samples were taken at t=0, 30, 60, 90 and 120 minutes, and fluorescence intensity was analyzed on a CytoFluor 2350 Fluorescence Measurement System (Millipore, Cambridge, Mass.). FD-3 concentrations transported were extrapolated from a standard curve (generated by diluting known concentrations of fluorescent tracer) and expressed as ng FD-3 transported/cm²/hour. Numerical values from individual experiments were pooled and expressed as mean ±standard error of the mean. Data shown are representative of at least three experiments.

### Measurement of Cloning Efficiency in Soft Agarose

Pa4 cells, Pa4-Raf1 cells and Pa4-Raf1 cells expressing full-length occludin or various occludin mutants were seeded a density of 1×10⁴ cells per 35-mm culture dish in 1 ml of 0.35% (wt/vol) low melting point agarose solution diluted with complete medium. Dishes were coated with 1 ml of 0.7% (wt/vol) low melting point agarose before cell plating, and 1 ml of overlay medium was added after cell plating. The dishes were incubated at 35° C. in 5% CC>2 and 95% air for 4 weeks. The overlay medium was changed every 3 days. After 4 weeks, the cells were stained with 3-[4,5-dimethylthiazol-2-yl]-2,5-diphenyltetrazolium bromide (MTT; 0.05 mg/ml). The stained dishes were photographed, and colonies >0.4 mm in diameter were counted and analyzed.

### Tumorigenicity in Nude Mice

The nude mice were used to assay tumorigenicity in vivo. Pa4 cells, Pa4-Raf1 cells and Pa4-Raf1 cells expressing fall-length occludin or various occludin mutants were grown to logarithmic growth phase, harvested, washed, and resuspended in PBS for injection. Each of the cell lines (1×10⁶ cells in 200 μl PBS) was injected subcutaneously into the flanks of four male nude mice. Tumor formation was monitored daily. All nude mice were sacrificed at six weeks, with tumors being isolated and analyzed.

### EXAMPLES

### Example 1

### Occludin Rescues the Epithelial Phenotype in Raf-1 Transformed Cells

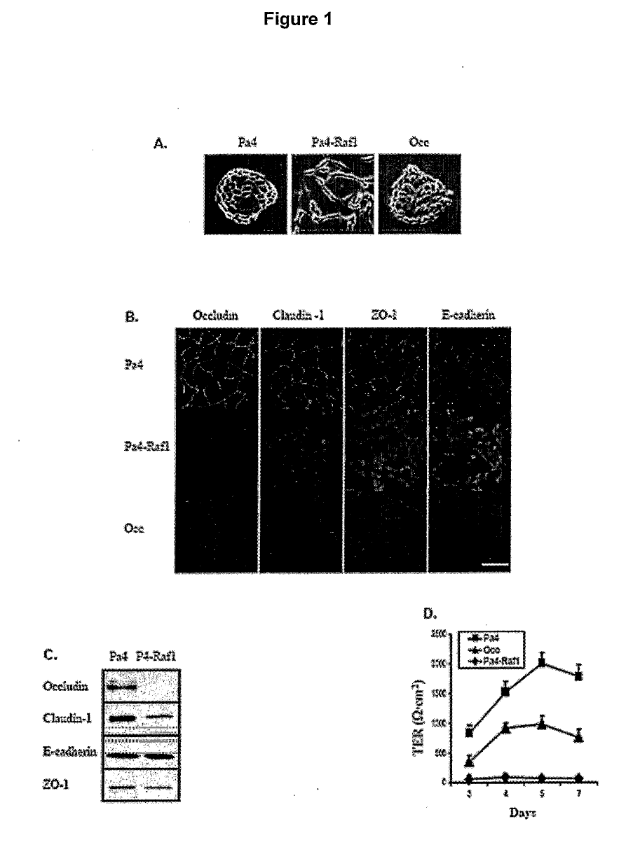

Introduction of constitutively active Raf1 results in the down-regulation of endogenous occludin in Pa4 cells that coincides with acquisition of a transformed phenotype. Introduction of the human occludin gene driven by a viral promoter induces forced expression of human occludin protein and reversal of the Raf1-induced oncogenic phenotype (Li & Mrsny, 2000). To investigate the role(s) of specific structural elements within the occludin protein on this Raf1-occludin dynamic, experiments were first performed to validate this rescue-of-function model in Pa4-Raf1 cells. Pa4 cells form tight colonies of polarized epithelial when grown on plastic surfaces in vitro (FIG. 1A), express junctional proteins such as occludin, claudin-1, ZO-1, and E-cadherin that were restricted to the apical neck region of the lateral plasma membrane: (FIG. 1B), and were readily detected in westem blots (FIG. 1C). Pa4 cells developed high values of transepithelial electrical resistance (TER) when cultured on semi-permeable membrane supports in vitro (FIG. 1D). Stable expression of constitutively active Raf1, however, transformed Pa4 cells into a fibroblast-like phenotype (FIG. 1A) that no longer expressed junctional proteins at the plasma membrane (FIG. 1B). Western blots showed that Pa4-Raf1 cells no longer expressed occludin and had reduced levels of claudin-1 and ZO-1 expression even though levels of E-cadherin appeared unaffected (FIG. 1C). Additionally, Pa4-Raf1 cells no longer developed measurable TER values when cultured on semi-permeable membranes (FIG. 1D). Consistent with previous studies (Li & Mrsny, 2000), forced expression of human occludin protein into Pa4-Raf1 cells (Occ) recovered epithelial morphology (FIG. 1A), junctional staining patterns (FIG. 1B), and barrier function (FIG. 1D) observed for Pa4 cells. Together, these results reaffirm that forced expression of full-length human occludin in Raf1-transformed cells can promote reacquisition of an epithelial phenotype and the formation of functionally intact TJs in Pa4 cells. Further experiments were then conducted to investigate the effect of specific occludin domain deletions in Raf1-transformed Pa4 cells.

### Example 2

### The Second Extracellular Loop of Occludin is Important for Rescue of Epithelial Cell Morphology

Occludin has been predicted to possess four transmembrane domains with two extracellular domains and cytosolic amino and carboxy termini (Furuse et al., 1993). A panel of occludin deletion constructs were made, all with a 10-amino acid myc tag engineered at the C-terminus to facilitate immunodetection of the transfected protein (FIG. 2A). Stable expression of constitutively active Raf1 in Pa4 cells (Pa4-Raf1) induced fibroblast-like growth characteristics in cells that lacked expression of occludin. ZO-1 was localized in patches at sites of cell-cell contact (FIG. 2B). Occludin constructs were then stably transfected into Pa4-Raf1 cells, and transgene expression was confirmed by immunodectection of the myc tag (data not shown). Phase contrast microscopy of cells grown on plastic in vitro demonstrated tightly clustered colonies in cells only following introduction of full-length occludin or construct lacking the first extracellular loop (OccΔL1). In both cases staining for ZO-1 and expressed occludin (either full-length or OccΔL1) co-localized in a highly organized “chicken wire” pattern typical of polarized epithelial cells (FIG. 2B). Introduction of occludin constructs lacking the N-terminal and extracellular domains (OccΔN) the C-terminal domain (OccΔC) or the second extracellular loop (OccΔL2) failed to rescue epithelial cell morphology (FIG. 2B upper panel). ZO-1 was frequently observed to co-localize with the myc-tag at cell-cell contacts in Pa4-Raf1 cells expressing OccΔN. However, Pa4-Raf1 cells expressing OccΔL2 forms of occludin more frequently showed intracellular distributions of ZO-1 and the myc-tag than those cells expressing OccΔN or OccΔC (FIG. 2B, lower panel). These results suggest that both the N-terminal and C-terminal domains of occludin are critical for rescue of epithelial cell morphology in Raf1-transformed Pa4 cells. In addition, it appears that, within the extracellular domain of occludin, the second loop but not the first loop is critical for this process.

### Example 3

### The Second Loop of Occludin is Important for Assembly of Junctional Complexes

More extensive junctional protein labeling studies comparing Pa4-Raf1 cells expressing the OccΔL1 and OccΔL2 mutants grown on permeable supports at high density demonstrated that claudin-1, ZO-1, and E-cadherin localized in the region of the apical junctional complex of OccΔL1 mutant cells (FIG. 3A). Importantly, this illustrates that OccΔL1 cells were able to form polarized monolayers with distinct junctional complexes (co-localization of ZO-1 and the myc-tag of the expressed occludin mutant protein). This pattern of junctional protein distribution in OccΔL1 cells was analogous to that observed in Pa4 cells expressing the full-length occludin (Occ) (FIG. 3B). In contrast to the OccΔL1 mutants, cells expressing the OccΔL2 mutant exhibited disorganized staining patterns of occludin (myc-tag), ZO-1, claudin-1 and E-cadherin. FIG. 3 illustrates disrupted staining patterns of these proteins in the plasma membrane and their localization in intracellular compartments.

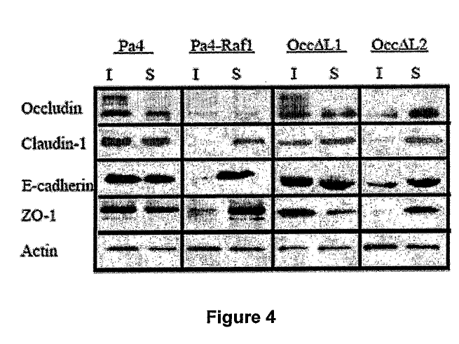

Non-ionic detergent insolubility is considered an indicator of protein incorporation into cytoskeleton-associated junctional complexes (Sakakibara et al., 1997). To complement the morphologic and confocal data (FIGS. 2&3), biochemical characterizations were performed of junctional protein expression and association with the non-ionic detergent Triton X-100 (TX-100) soluble (S) or insoluble (I) fractions of cell homogenates prepared from Pa4, Pa4-Raf1, OccΔL1 or OccΔL2 cells (FIG. 4). Hyperphosphorylated occludin expressed in Pa4 cells is mostly associated with the I fraction of these cells while claudin-1, ZO-1, and E-cadherin are equally present in the I and S fractions. Introduction of constitutively active Raf1 (Pa-4Raf1) results in loss of normal junction organization exemplified by the total loss of expressed occludin and a shift of claudin-1, ZO-1, and E-cadherin out of the I fraction of these cells (FIG. 4). Occludin protein lacking the first loop (OccΔL1) expressed in Pa4-Raf1 cells was found to distribute between I and S fractions, similarly to that observed for Pa4 cells. Importantly, the solubility profile for claudin-1, ZO-1, and E-cadherin in the OccΔL1 cells greatly resembled that of the parental Pa4 epithelial cell line (FIG. 4). In contrast, Pa4-Raf1 cells expressing OccΔL2 demonstrated proportionately less claudin-1, ZO-1 and E-cadherin in the detergent-insoluble fractions, and this pattern closely resembled that of oncogenic Pa4-Raf1 cells (FIG. 4). Interestingly, expressed OccΔLZ was almost exclusively in the S fraction. Overall, the differences in solubility profiles observed for the OccΔL1 and OccΔL2 cell lines are consistent with the differences in cell morphology and junctional staining patterns.

### Example 4

### The Second Loop of Occludin is Important for TJ Barrier Function

Various studies have reported that the extracellular loops of occludin are important for regulation of TJ barrier function in epithelial monolayers. Pa4-Raf 1 cells expressing wild type human occludin (Occ) formed monolayers with transepithelial electrical resistance (TER) values of >900 O-cm² after 5 days of culture on semi-permeable filter supports in vitro (FIG. 5A). While Pa4-Raf1 cells expressing the OccΔL2 mutant did not produce TER values above background (<80 Ω-cm²), introduction of OccΔL1 mutant produced TER values >500 Ω-cm² on day 5 of culture. While TER in these OccΔL2 cells is lower than that observed in Pa4 cells transfected with full length occludin, the TER values of >5000-cm² support development of functional TJs. Since electrical and solute barrier properties of epithelial cells can be disconnected (Balda et al., 1996), the effect of the OccΔL1 and OccΔL2 mutants on FD-3 (3 kDa FITC-dextran) flux as an indicator of paracellular permeability was also investigated. FD-3 flux across Pa4 cells showed highly restricted movement of this paracellular permeability marker (FIG. 5B). Pa4-Raf1 cells corrected with the introduction of full-length human occludin had FD-3 flux rates of −1,500 ng/cm²/hr while forced expression of OccΔL1 in Pa-4Raf1 cells resulted in somewhat higher flux rates (˜3-fold higher) (FIG. 5B). As expected, forced expression of OccΔL2 in Pa4-Raf1 cells did not exhibit any restriction to FD-3 flux (data not shown). Together these results suggest that the second loop of occludin is essential for TJ barrier function, but expression of a mutant with only the second loop (OccΔL1) is not sufficient to completely rescue TJ barrier function.

### Example 5

### The Second Loop of Occludin is Required for Anchorage-Dependent Growth in Soft Agarose

Loss of anchorage-dependent growth on soft agarose is considered an indicator of oncogenic transformation. In the case of Pa4 cells, transformation with constitutively active Raf1 has been shown to confer the capacity for anchorage-independent growth, while over-expression of exogenous occludin was observed to restore anchorage dependence (Li & Mrsny, 2000). To investigate whether specific occludin domains are required to suppress anchorage-independent growth, Pa4-Raf1 cells expressing various human occludin mutants or full-length human occludin were subjected to growth assays in soft agar (FIG. 6A). Pa4-Raf1 cells expressing the OccΔC, OccΔN and OccΔL2 mutants formed numerous colonies on soft agarose, averaging 105, 100, and 80 colonies, respectively. In contrast, expression of full-length occludin (Occ) or the OccΔL1 mutant significantly inhibited growth on soft agarose, with only 5 and 10 colonies on average, respectively (FIG. 6B). These results suggest that the second loop of occludin is essential for suppression of anchorage-independent growth, an indicator of oncogenic transformation.

### Example 6

### The Second Loop of Occludin is Required for Suppression of Raf-Mediated Tumor Formation in Nude Mice

To better establish the potential role played by the second loop of occludin on Pa-4 cell proliferation, we assessed in vivo growth characteristics of Pa4-Raf 1 expressing various occludin mutants. Pa4-Raf1 cells expressing full-length human occludin or various mutants (FIG. 2A) were injected subcutaneously into the flanks of nude mice. Tumor formation was assayed by visual inspection. Injection of Pa4 cells (data not shown) or Pa4-Raf1 cells expressing either full-length human occludin (Occ) (FIG. 7A) or the OccΔL1 (data not shown) failed to produce tumors 6 weeks post injection (FIG. 7B). Oppositely, injection of Pa4-Raf1 cells expressing OccΔL2 (FIG. 7A) or OccΔC or OccΔN, or Pa4-Raf1 cells (data not shown) did produce large visible tumors (FIG. 7B).

Significant differences were not observed in the amount of tumor growth resulting from subcutaneous injection of these various cells lines (FIG. 7B), although these studies were performed with a limited number of animals and injection sites. Together these results extend the previous in vitro studies by demonstrating that expression of exogenous occludin (or occludin lacking only the first extracellular loop) can suppress Raf1-induced tumor growth in nude mice, and the second loop of occludin is required for growth suppression of Raf1 transformed Pa4 cells.

### Example 7

### Active Raf1 Down-Regulates Tight Junction Protein Gene Expression

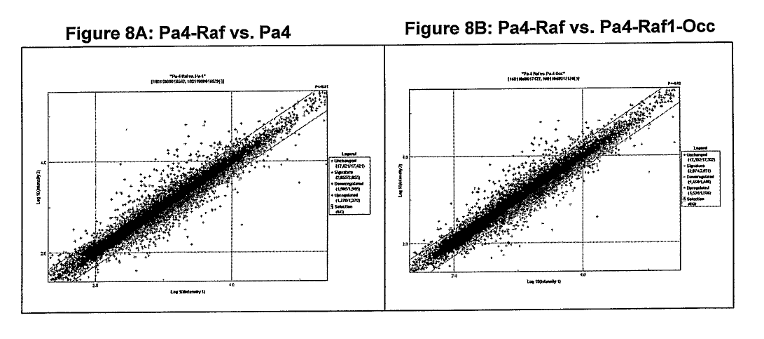

Based upon internal quality control data, differential expression values greater than 1.5 obtained with Agilent Oligonucleotide microarrays are likely to be significant. Using this benchmark, we compared microarray data for entire rat genome obtained from Pa-4 cells before and after stable transfection with constitutively active Raf1 (Pa4-Raf1). Comparison of mRNA abundance levels between Pa4 and Pa4-Raf1 cells demonstrated that a large number of genes had been up- or down-regulated by the actions of Raf1 (FIG. 8A). Genes affected by active Raf1 in Pa4 cells included transcription factors, response elements, regulatory molecules, growth factor receptors, cell-cell contact proteins, and cytoskeletal elements that were consistent with those identified in other studies examining epithelial cell gene modulation associated with Ras/Raf/MAP kinase pathway activation. The strongest mRNA abundance profile shifts occurred in genes associated with cell polarity, proliferation and sensitivity to apoptotic stimuli. Numerous genes identified by the present studies have been previously reported as being targets of cell regulation in other cancer cell microarray studies.

Of particular significance, TJ-associated protein mRNA abundance levels that were significantly reduced when comparing Pa4 to Pa4-Raf1 cells include the following: occludin (−1.8 fold), claudin-7 (−12 fold), claudin-3 (−2.6 fold), claudin-1 (−2.5 fold). These results are consistent with changes in mRNA abundance levels of prostatic epithelial cells associated with tumorigenesis and our previous studies showing decreased occludin mRNA and expression levels in Pa4 cells following activation of Raf1. More recent studies examining Pa4-Raf1 cells have identified a decrease in both mRNA and protein levels for claudin-1. These findings are important from the perspective that down-regulation of TJ components is associated with epithelial cell transformation events.

### Example 8

### Occludin Rectifies Raf1-Driven Gene Expression

Forced expression of occludin in Pa4-Raf1 cells (producing Pa4-Raf1-Occ cells) by a transfection approach does not affect the constitutively active Raf1 kinase activity in these cells. For these studies, human occludin, rather than rat occludin, was introduced as a means of verifying that spontaneous re-expression of endogenous occludin protein had not occurred. Forced expression of human occludin can reverse the oncogenic phenotype of Pa4-Raf1 cells assessed by growth in soft agar and subcutaneous injection into nude mice. Comparison of mRNA abundance level in Pa4-Raf1 to those of Pa4-Raf1-Occ cells showed a remarkable reversal of essentially all of the genomic changes induced by introduction of active Raf1 into these Pa-4 cells (FIG. 8B).

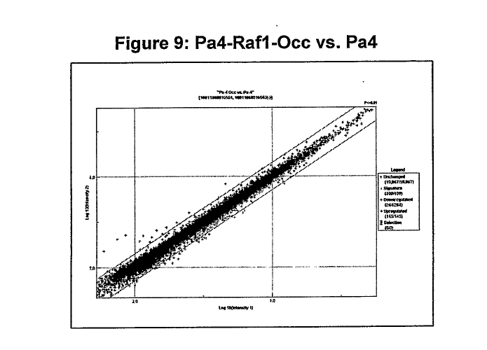

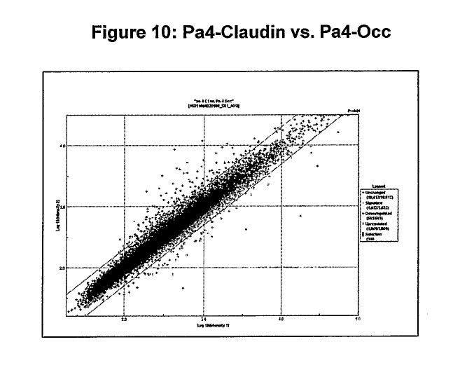

Comparison of abundance changes for specific mRNAs for Pa-4 cells (prior to introduction of active Raf1) with those for Pa4-Raf1-Occ (after forced human occludin expression) were compared as a means of assessing the reversible nature of genes regulated through this unanticipated Raf1-Occludin dynamic (FIG. 9). Recovery of TJ-related protein genes provides one aspect of this dynamic that is particularly pertinent to these studies. Further, all of the identified genes showing the greatest alteration in mRNA abundance due to Raf1 activation were reversed following forced expression of human occludin.

### Example 9

### Claudin-1 does not Rectify Raf1-Driven Gene Expression

Numerous studies have described an essential role for claudin proteins in establishing and maintaining functional TJ structures. Although activation of Raf1 signaling events can lead to mammary gland carcinogenesis, inhibition of MAPK activity in a series of breast cancer cell lines failed to recover depressed protein levels of critical TJ proteins such as occludin and claudin-1, suggesting that the Ras/Raf/MAPK pathway may not be involved in dys-regulation of TJ structures in these breast cancer cells. While these authors have demonstrated claudin-1 can induce apoptosis, it appears that non-Raf-regulated events associated with claudin-1 protein expression may be more critical than events associated with occludin expression in breast cancer cells. Previous studies where claudin-1 was expressed in Pa4-Raf1 cells (Pa4-Raf1-Claudin-1) showed a lack of correction of events associated with Raf1-induced EMT. Comparison of microarray analysis data obtained for Pa4-Raf1-Claudin 1 cells to other array sets shows that forced expression of this TJ protein fails to affect correction of gene modifications produced by activation of Raf1 in Pa-4 cells. It is possible that tissue-to-tissue (or cell-to-cell) variability may be an important factor in epithelial cell responses to Raf1 activation and occludin rectification. It is also possible that claudin family members other than claudin-1 may be more actively involved in the Raf1/Occ dynamic. In this regard, claudin-3 and claudin-7 are affected by malignant transformation of prostate epithelial cells and we observed these same two claudin proteins to be strongly regulated in the present studies.

### Example 10

### Correction of Raf1-Induced Oncogenic Effects in Epithelial Cells

The present studies support a dynamic relationship between Raf1 and occluding expression, which in turn determines epithelial cell differentiation. Dysregulation of this pathway is associated with oncogenic growth of epithelial cells. Many factors participate in this regulatory process. Based on previous studies such factors include growth factors, anti-apoptosis molecules, etc. For example, mRNA abundance levels of the tumor-associated calcium signal transducer 1 gene is modified four-fold by the Raf/Occ dynamic while the vascular adhesion molecule Vcam1 is modified 17-fold and the vascular endothelial growth factor C gene transcription is affected 3-fold. Abundance levels for a gene termed REX-3, previously shown to be affected by retinoic acid in F9 teatocarcinoma cells (Faria 1998), were up-regulated 37-fold by Raf1 and down-regulated by occludin to an equivalent extent.

Of particular interest, however, are several genes that have not been shown to directly affect growth factor signaling and cell survival pathways. In this regard, nearly fifty mRNA transcripts were highly regulated that could be potential targets for cancer therapeutic strategies. Examination of a few of these candidate genes supports the potential of their role in cancer cell survival and metastasis. For example, the Raf1/Occ dynamic strongly regulates (30-fold) transcriptional regulation of the osteopontin gene. Osteopontin (or sialoprotein) is a secreted, phosphorylated, glycoprotein with cytokine and adhesion protein-like qualities that has been shown to be up-regulated by and associated with tumorigenic and metastatic events (Denhardt et al, 2003; Rittling et al., 2004). Osteopontin has also been implicated in tumor metastasis and angiogenesis (Asou et al., 2001; Nemoto et al., 2001). Additionally, a recent finding suggests that the cell-surface water channel aquaporin-1 is essential for new vessel growth (Saddoun et al., 2005); Raf1 induced a nearly 4-fold increase in aquaporin-1 mRNA levels, which was corrected by occludin expression. One of the most highly regulated genes affected by the Raf1/Occ dynamic was a growth factor binding protein, down-regulated in Pa-4 cells 38-fold by Raf1 and up-regulated 35-fold in Pa4 Raf1 cells by occludin.

A series of specific transcription regulators, such as the H2.0-like homeo box gene and the MADS box transcription enhancer factor 2, are controlled by the Raf1/Occ dynamic, making these potential targets for therapeutic intervention for cancers. Other potential cancer targets identified in the Raf1/Occ dynamic that would not be anticipated include the following from Table 1 below:

TABLE 1

| Gene | Acronym | Raf1 regulation |
|---|---|---|
| serine (or cysteine) proteinase inhibitor (nexin, | Serpine1 | up-regulated 5 fold |
| plasminogen activator inhibitor type 1) | | |
| Neural adhesion molecule | Ncam | up-regulated 3 fold |
| Calcitonin receptor activity modifying protein 1 | Ramp1 | up-regulated 2 fold |
| Regulator of G-protein signaling protein 2 | Rgs2 | up-regulated 2 fold |
| Pyrimidigenic receptor P2Y, G-protein | P2ry6 | up-regulated 4 fold |
| coupled, 6 | | |
| PKC and casein kinase substrate in neurons | Pacsin2 | down-regulated 2 fold |
| Serine protease 8, prostasin | Prss8 | down-regulated 8 fold |
| Plasminogen activator inhibitor 2 type A | Pai2a | down-regulated 3 fold |
| Neural pentraxin1 | Nptx1 | down-regulated 6 fold |
| Rattus norvegicus small inducible cytokine | Scyb2 | up-regulated 6 fold |
| subfamily, member 2 | | |
| WAP four-disulfide core domain 1 (serine protease | Wfdc1 | down-regulated 8 fold |
| inhibitor) | | |
| annexin A8 | Anx8 | down-regulated 15-fold |
| monocyte neutrophil elastase inhibitor | Serpinb1 | down-regulated 4-fold |
| forkhead protein | Foxf1a | up-regulated 3-fold |
| glycogenin 2 | Glyg2 | down-regulated 7-fold |
| MAP kinase phosphatase 6 | Mkp6 | down-regulated 4-fold |
| Actin-binding protein adseverin D5 | Adsd5 | down-regulated 3-fold |
| SH2-containing leukocyte protein 65 | Sclp65 | down-regulated 6-fold |
| disintegrin and metalloprotease domain 8 | Dmd8 | down-regulated 6-fold |
| Tetraspanin NET-2 | Tns2 | up-regulated 7-fold |
| Stanniocalcin-2 | Snc2 | down-regalated 3-fold |
| Translin-associated factor X | TafX | up-regulated 6-fold |

All of these gene modifications were corrected by the introduction of occludin. Thus, introduction, rectification, or modulation of function of these various gene products and/or their functions provide unique and unanticipated opportunities for cancer therapies either separately or as an adjuvant to occludin protein correction. Some of these targets also represent potential methods to ameliorate some of the pathologies associated with cancer such as wasting, bone resorption, etc.

### Example 11

### Oncogenic Raf1 Induces Epithelial Cell Transformation by Suppressing Occludin Promoter and Activating Slug

Disruption of the AJC with the loss of concomitant junctional protein expression is a hallmark of cancer cell invasion and metastasis. Activation of the Ras-Raf signaling pathway has been implicated in a subgroup of epithelial derived cancers. Thus, our studies examined mechanisms by which oncogenic Raf-1 induces epithelial mesenchymal transition (EMT) and transformation into an oncogenic phenotype. Using a rat salivary gland epithelial cell line (Pa4), we have demonstrated that constitutive expression of the Raf-1 oncogene in Pa4 epithelial cells disrupts functional TJs and transforms the epithelial phenotype into an oncogenic phenotype by downregulating occludin expression. To better characterize the mechanism by which Raf-1 activity influences occludin expression and EMT, we analyzed the influence of Raf-1 on the occludin promoter activity. A 1853-base pair (bp) fragment of the occludin promoter was isolated and a series of deletion constructs were generated. Transfection of these constructs into Raf-1 transformed cells revealed that a minimal segment of the occludin promoter (−50/+240) was repressed by Raf-1. Furthermore, oncogenic Raf-1 induced upregulation of the transcription repressor “Slug” (a zinc-finger-containing gene) with subsequent downregulation of occludin. These events correlated with epithelial cell transformation. Interestingly, in contrast to other studies analyzing EMT, E-cadherin expression remained unchanged. siRNA-mediated suppression of Slug in Raf-1 transformed cells resulted in enhanced occludin expression and morphologic changes consistent with transition to an epithelial phenotype. These findings support a role of Slug in mediating Raf-1 induced suppression of occludin and subsequent EMT.

While the foregoing embodiments have been set forth in considerable detail for the purpose of making a complete disclosure of the invention, it will be apparent to those of skill in the art that numerous changes can be made in such details without departing from the spirit and the principles of the disclosure. Accordingly, the disclosure should be limited only by the following claims.

### REFERENCES

Ando-Akatsuka, Y., Saitou, M., Hirase, T., Kishi, M., Sakakibara, A., Itoh, M., Yonemura, S., Furuse, M. & Tsukita, S. (1996). J Cell Biol, 133, 43-7.
Asou Y, Rittling S R, Yoshitake H, Tsuji K, Shinomiya K, Nifuji A, et al. Osteopontin facilitates angiogenesis, accumulation of osteoclasts, and resorption in ectopic bone. Endocrinology 2001; 142(3):1325-32.
Balda, M. S., Flores-Maldonado, C., Cereijido, M. & Matter, K. (2000). J Cell Biochem, 78, 85-96.
Balda, M. S., Garrett, M. D. & Matter, K. (2003). J Cell Biol, 160, 423-32. Balda, M. S. & Matter, K. (2000). Embo J, 19, 2024-33.
Balda, M. S., Whitney, J. A., Flores, C., Gonzalez, S., Cereijido, M. & Matter, K. (1996). I Cell Biol, 134, 1031-b 49.
Bamforth, S. D., Kniesel, U., Wolburg, H., Engelhardt, B. & Risau, W. (1999). J Cell Sci, 112 (Pt 12), 1879-88.
Betanzos, A., Huerta, M., Lopez-Bayghen, E., Azuara, E., Amerena, J. & Gonzalez-Mariscal, L. (2004). Exp Cell Res, 292, 51-66.
Chen, Y., Merzdorf, C., Paul, D. L. & Goodenough, D. A. (1997). I Cell Biol, 138, 891-9.
Denhardt D T, Mistretta D, Chambers A F, Krishna S, Porter J F, Raghuram S, et al. Transcriptional regulation of osteopontin and the metastatic phenotype: evidence for a Ras-activated enhancer in the human OPN promoter. Clin Exp Metastasis 2003; 20(1):77-84.
Faria T N, LaRosa G J, Wilen E, Liao J, Gudas L J. Characterization of genes which exhibit reduced expression during the retinoic acid-induced differentiation of F9 teratocarcinoma cells: involvement of cyclin D3 in RA-mediated growth arrest. Mol Cell Endocrinol 1998; 143(1-2):155-66.
Furuse, M., Fujita, K., Hiiragi, T., Fujimoto, K. &Tsukita, S. (1998a). J Cell Biol, 141, 1539-50.
Furuse, M., Hirase, T., Itoh, M., Nagafuchi, A., Yonemura, S. & Tsukita, S. (1993). J Cell Biol, 123, 1777-88.
Furuse, M., Itoh, M., Hirase, T., Nagaruchi, A., Yonemura, S. & Tsukita, S. (1994). J Cell Biol, 127, 1617-26.
Furuse, M., Sasaki, H., Fujimoto, K. & Tsukita, S. (1998b). I Cell Biol, 143, 391-401.
Hirase, T., Staddon, J. M., Saitou, M., Ando-Akatsuka, Y., Itoh, M., Furuse, M., Fujimoto, K., Tsukita, S. & Rubin, L. L. (1997). J Cell Sci, 110 (Pt 14), 1603-13.
Hoover, K. B., Liao, S. Y. & Bryant, P J. (1998). Am J Pathol, 153, 1767-73.
Itoh, M., Furuse, M., Morita, K., Kubota, K., Saitou, M. & Tsukita, S. (1999). J Cell Biol, 147, 1351-63.
Itoh, M., Nagaruchi, A., Moroi, S. & Tsukita, S. (1997). I Cell Biol, 138, 181-92.
Kimura, Y., Shiozaki, H., Hirao, M., Maeno, Y., Doki, Y., Inoue, M., Monden, T., Ando-Akatsuka, Y., Furuse, M., Tsukita, S. & Monden, M. (1997). Am J Pathol, 151, 45-54.
Kominsky, S. L., Argani, P., Korz, D., Evron, E., Raman, V., Garrett, E., Rein, A., Sauter, G., Kallioniemi, O. P. & Sukumar, S. (2003). Oncogene, 22, 2021-33.
Lacaz-Vieira, F., Jaeger, M. M., Farshori, P. & Kachar, B. (1999). J Membr Biol, 168, 289-97.
Li, D. & Mrsny, R J. (2000). J Cell Biol, 148, 791-800.
Martin-Padura, I., Lostaglio, S., Schneemann, M., Williams, L., Romano, M., Fruscella, P., Panzeri, C., Stoppacciaro, A., Ruco, L., VIIIa, A., Simmons, D. & Dejana, E. (1998). J Cell Biol, 142, 117-27.
Matter, K. & Balda, M. S. (1998). J Cell Sci, 111 (Pt 4), 511-9. Matter, K. & Balda, M. S. (2003). Nat Rev Mol Cell Biol, 4, 225-36.
Medina, R., Rahner, C., Mitic, L. L., Anderson, J. M. & Van Itallie, C. M. (2000). J Membr Biol, 178, 235-47.
Michl, P., Barth, C., Buchholz, M., Lerch, M. M., Rolke, M., Holzmann, K. H., Menke, A., Fensterer, H., Giehl, K., Lohr, M., Leder, G., Iwamura, T., Adler, G. & Gress, T. M. (2003). Cancer Res, 63, 6265-71.
Morita, K., Furuse, M., Fujimoto, K. & Tsukita, S. (1999). Proc Natl Acad Sci USA, 96, 511-6.
Nakamura, T., Blechman, J., Tada, S., Rozovskaia, T., Itoyama, T., Bullrich, F., Mazo, A., Croce, C. M., Geiger, B. & Canaani, E. (2000). Proc Natl Acad Sci USA, 97, 7284-9.
Nemoto H, Riftling S R, Yoshitake H, Furuya K, Amagasa T, Tsuji K, et al. Osteopontin deficiency reduces experimental tumor cell metastasis to bone and soft tissues. J Bone Miner Res 2001; 16(4):652-9.
Rittling S R, Chambers A F. Role of osteopontin in tumour progression. Br J Cancer 2004; 90(10):1877-81.
Saadoun S, Papadopoulos M C, Hara-Chikuma M, Verkman A S. Impairment of angiogenesis and cell migration by targeted aquaporin-1 gene disruption. Nature 2005; 434(7034):786-92.
Saitou, M., Furuse, M., Sasaki, H., Schulzke, J. D., Fromm, M., Takano, H., Noda, T. & Tsukita, S. (2000). Mol Biol Cell, 11, 413142.
Sakakibara, A., Furuse, M., Saitou, M., Ando-Akatsuka, Y. & Tsukita, S. (1997). J Cell Biol, 137, 1393-401.
Sanders, S. E., Madara, J. L., McGuirk, O. K., Gelman, D. S. & Colgan, S. P. (1995). Epithelial Cell Biol, 4, 25-34.
Tobioka, H., Isomura, H., Kokai, Y., Tokunaga, Y., Yamaguchi, J. & Sawada, N. (2004). Hum Pathol, 35, 159-64.
Tsukita, S. & Furuse, M. (1999). Trends Cell Biol, 9, 268-73.
Van Itallie, C. M. & Anderson, J. M. (1997). J Cell Sci, 110 (Pt 9), 1113-21.
Wittchen, E. S., Haskins, J. & Stevenson, B. R. (1999). J Biol Chem, 274, 35179-85.
Wang, Z., Mandell, K., Parkos, C., Mrsny, R. & Nusrat, A. (2005) Oncogene, 24, 4412-4420.
Wong, V. & Gumbiner, B. M. (1997). J Cell Biol, 136, 399-409.

We claim:

1. A method for identifying modulators of cellular tight junctions comprising:
(a) contacting transformed epithelial or transformed endothelial cells with a test compound;
(b) determining formation of functional tight junctions by the transformed cells contacted with the test compound; and
(c) selecting the test compound that increases formation of tight junctions compared to a control compound, wherein the test compound increases formation of tight junctions by modulating occludin activity of the transformed cells.

2. The method of claim 1, wherein the selected compound induces a polarized phenotype characteristic of functional epithelia.

3. The method of claims 1 or 2, wherein the test compound mimics occludin activity.

4. The method of claims 1 or 2, wherein the test compound upregulates occludin expression.

5. The method of claims 1 or 2, wherein the test compound comprises a polynucleotide or a polypeptide.

6. The method of claims 1 or 2, wherein the transformed cells comprise Pa4-Raf1 cells.

7. The method of claims 1 or 2, wherein the test compound interacts with a second loop of occludin.

8. A method for identifying modulators of epithelial to mesenchymal transformation comprising:
(a) contacting occludin or a fragment thereof with a test compound;
(b) determining whether the test compound interacts directly or indirectly with occludin or the fragment thereof; and
(c) selecting the test compound that interacts with occludin's second loop and reverses epithelial to mesenchymal transformation phenotype changes in transformed cells.

9. The method of claim 8, wherein the selected compound induces a polarized phenotype characteristic of functional epithelia in transformed cells.

10. The method of claim 8, wherein the test compound comprises a polynucleotide, polypeptide, or a small organic molecule.

11. The method of claim 8, wherein the transformed cells are transformed by Raf1.

12. A method for modulating Raf1 induced transformation comprising:
contacting a cell transformed by Raf1 with a composition that induces expression of occludin in the transformed cell, reversing epithelial to mesenchymal transformation phenotype changes due to Raf1.

13. A method of treating epithelial or endothelial cell transformation comprising:
contacting a transformed epithelial or endothelial cell with a composition comprising an occludin modulator in an amount sufficient to reverse epithelial to mesenchymal transformation phenotype changes.

14. The method of claim 13, wherein the epithelial to mesenchymal transformation phenotype changes are due to Raf1.

15. A composition for treating skin cancer comprising:
a vector encoding a recombinant occludin polypeptide comprising a second loop and carboxy terminus of occludin in an amount sufficient to reverse epithelial to mesenchymal transformation phenotype changes due to Raf1; and
a pharmaceutically acceptable carrier or excipient.

16. A composition for treating epithelial or endothelial cell transformation comprising:
an amount of an occludin modulator effective to reverse epithelial to mesenchymal transformation phenotype changes due to Raf1 in a host.

17. The composition of claim 16, wherein the host comprises a mammal.

18. The compositions of claim 16, wherein the occludin modulator comprises a polynucleotide, polypeptide, small organic molecule or combinations thereof.

19. The composition of claim 16 further comprising a second therapeutic agent.

20. The method of claim 19, wherein the second therapeutic agent is a chemotherapeutic agent.
